(19) United States
(12) Patent Application Publication
(10) Pub. No.: US 2026/0345045 A1
(43) Pub. Date: Nov. 5, 2026

(54) Device for Sensing Magnetic Signals Generated by a Beating Heart

(71) Applicant: Robert Bosch GmbH, Stuttgart (DE)

(72) Inventors: Florian Dolde, Stuttgart (DE); Frederik Schaal, Reutlingen (DE); Florian Krist, Stuttgart (DE); Riccardo Cipolletti, Geislingen An Der Steige (DE); Eckhard Wehrse, Stuttgart (DE)

(21) Appl. No.: 19/108,086

(22) PCT Filed: Aug. 18, 2023
(86) PCT No.: PCT/EP2023/072813

§ 371 Date: Feb. 28, 2025

(30) Foreign Application Priority Data
Sep. 9, 2022 (DE) 10 2022 209 429.8

(51) Int. Cl. A61B 5/243; A61B 5/00; G01R 33/26; G01R 33/28

(52) U.S. Cl. CPC A61B 5/243; A61B 5/6891; G01R 33/26; G01R 33/281; A61B 2562/046; G01R 33/282

(57) ABSTRACT

A device for sensing magnetic signals produced by a beating heart includes a first assembly of at least two nitrogen vacancy centers, NV, magnetometer units. The first assembly has a first main extension plane. The device further includes a second assembly of at least two NV magnetometer units. The second assembly has a second main extension plane. The first main extension plane is not parallel to the second main extension plane.

The present invention relates to a device for sensing magnetic signals generated by a beating heart

### BACKGROUND OF THE INVENTION

Optically pumped or diamond-based quantum sensors based on NV centers are particularly suitable as sensors for measuring very small magnetic field strengths. DE 10 2022 204 526.2 describes a magnetometer that utilizes optically pumped and optically detected magnetic resonance (ODMR). It is thereby exploited that, under the influence of an external magnetic field, the energy levels of certain spin states of unpaired electrons are split, the so-called Zeeman effect. The splitting of the energy levels results in changed transitions during relaxation from excited states, which can then be measured, for example, by optical excitation and frequency-dependent detection of the resulting fluorescence radiation or by observation of optical properties such as the absorption of light. From the measured optical parameters, the magnetic field strength may then in turn be inferred.

### DISCLOSURE OF THE INVENTION

According to the invention, a device for sensing magnetic signals generated by a beating heart is proposed with the features of claim 1. Advantageous configurations are the subject matter of the dependent claims and the following description.

A magnetocardiogram (abbreviated MCG) is the recording and representation of the magnetic field of the heart that arises from the electrophysiological activity of the myocardial cells. In the context of the invention, a contactless, passive approach for long-term monitoring of the human heart with high resolution is presented. This is determined by nitrogen vacancy magnetometers (so-called NV magnetometers) in a geometric arrangement, in particular for vector gradiometry.

Specifically, a device for sensing magnetic signals generated by a beating heart is now presented having a first assembly of at least two NV magnetometer units having a first main extension plane and a second assembly of at least two NV magnetometer units having a second main extension plane, wherein the first main extension plane is not parallel to the second main extension plane. Such a device may also be referred to as a magnetocardiograph. Thus, different directional components of the magnetic signals can be better resolved. This has technical advantages because magnetic field lines are always distributed in a circular manner around a flowing current. Thus, coverage is greatly improved by spatially distributed sensors. This extends the possibilities of signal processing, e.g. in particular to include integration. Also, a measurement may continue to be carried out effectively even when a user is moving.

To provide the widest possible coverage, the first main extension plane and the second main extension plane in one exemplary embodiment include an angle of at least 25°, at least 30°, at least 45°, or at least 60°. In one exemplary embodiment, the first major extension plane and the second major extension plane include an angle of 80°-90°. This allows magnetic signals to be recorded for any rotational position of the heart about an axis of rotation, e.g., when a user is standing or lying down.

The main extension plane of an assembly is in particular the plane in which a projection of the assembly comprises the largest surface area.

In one exemplary embodiment, the device comprises a third assembly consisting of at least two NV magnetometer units having a third main extension plane, wherein the third main extension plane is not parallel to the first and not parallel to the second main extension plane. This allows magnetic signals to be captured for any spatial orientation of the heart. In one exemplary embodiment, there is an angle between two of the first, second and third major extension planes, respectively, selected from at least 25°, at least 30°, at least 45°, at least 60°, or 80°-90°.

In one exemplary embodiment, the device comprises a further assembly consisting of at least one NV magnetometer unit which serves to detect a background magnetic field. By directly measuring and considering the background magnetic field, the magnetic fields of the heart can be measured even more accurately.

In one exemplary embodiment, the first and/or second and/or third and/or further assembly is embedded in a body selected from a building wall, a building ceiling, a building floor and a furnishing. Existing circumstances, in particular, can be exploited to sense the spatial magnetic field of the heart, for example, adjacent apartment walls or roof slopes. For optimal noise suppression or background field detection, on the other hand, assemblies can be placed on the sides of the home where the highest level of magnetic noise can be detected, e.g., towards busy roads or high power lines.

A particular advantage of the NV sensors is their size, specifically of the sensor medium. For the application, the active measurement volume should be small compared to the object (heart) to be measured, otherwise integration will occur over large portions of the signal due to the surface coverage, and thus the signal may disappear because the integral is zero. The smaller the active measurement volume is compared to the heart, the better the signal detection will be. NV sensor technology has a very small active sensor volume. This capability for creating small designs also allows the sensors to be used in a geometric arrangement. In particular, very high resolution arrangements are possible due to the very small active sensor volume. This also enables easy integration into the body or into objects.

Diamond NV magnetometers are based on reading out magnetic resonances from specific defect centers in diamond, in particular nitrogen vacancies (NV), which occur as impurities in the carbon lattice of diamond and can also be introduced in a targeted manner. If the NV center is optically excited in the ground state, e.g., by radiating in a pump laser beam having a suitable wavelength (in this case in the green wavelength range, e.g., at 532 nm for off-resonance excitation), the electrons are lifted from the triplet ground state to the excited triplet state and relax while emitting fluorescence light in the red wavelength range at 650-800 nm (637 nm=zero phonon line). Since the probability of non-spin-conserving transitions from the spin state is higher when the spin quantum number mₛ=±1, continuous excitation pumping ensures that the NV centers are largely hyperpolarized in the spin state mₛ=0.

There is an energy difference between the mₛ=0 and mₛ=±1 spin states in the ground state, which in this case is about 2.87 GHz. Thus, if microwave radiation is radiated into the diamond in addition to the optical excitation, there is a dip in the red fluorescence at this resonance frequency of 2.87 GHz, because the spin-polarized electrons are lifted by the microwave field from mₛ=0 to the mₛ=±1 ground state and excited from there by the pump light to the mₛ=±1 excited state. From there, however, primarily non-radiative transitions and weak infrared fluorescence transitions occur via the singlet state, and the fluorescence in the red range ceases.

If an external magnetic field is present, the so-called Zeeman effect leads to the splitting of the otherwise energetically equal mₛ=±1 triplet levels into energetically equidistant Zeeman levels. Plotting the fluorescence against a frequency spectrum of the microwave excitation then reveals two dips in the fluorescence spectrum, the frequency spacing of which is linearly proportional to the magnetic field strength of the external magnetic field. The magnetic field sensitivity is primarily defined by the minimally resolvable frequency shift and can reach 1 pT/√Hz or less. Because the nitrogen vacancy center in the single-crystal diamonds has four possible ways to position itself in the crystal lattice, the presence of a directed magnetic field causes the nitrogen vacancy centers present in the crystal to react differently to the external magnetic field depending on their position in the crystal. As a result, four pairs of fluorescence minima may appear in the spectrum, from the shape and position of which both the magnetic field strength as an amount and the direction of the external magnetic field are clearly determinable.

In order to enable vectorial magnetic field measurements, the device in one embodiment comprises a device for generating a substantially homogeneous bias magnetic field in the area of the magnetometer units or their sensor media, wherein the bias magnetic fields of different NV magnetometer units are expediently different. The device can also be integrated in the body. This may be a Helmholtz coil assembly, wherein at least the sensor medium is disposed within the Helmholtz coil assembly. It can also be other equipment such as a simple coil, a long coil, permanent magnet solutions such as in a Hallbach array, etc.

Cardiac signals have a magnetic signature at a distance of some cm with an amplitude of (only) single digit to double digit picotesla (pT), whereas, for example, the Earth's magnetic field in central Europe is approximately 50 μT (microtesla), i.e., it is more powerful by a factor of 10⁶. However, even such small field strengths are resolvable with a high level of precision for a long period of time with the proposed technology. For example, a magnetic shield or a gradiometer circuit may be used for this purpose.

High resolution detection of the accurate heart signal can be used to detect a variety of diseases such as permanent atrial fibrillation and paroxysmal atrial fibrillation. Thus, a heart attack and subsequently a stroke (particularly after an unrecognized heart attack) can be prevented. Further, the invention is suitable for early detection of an ST segment elevation infarction, a different kind of elevation infarction, a pulmonary embolism, an AV nodal reentry tachycardia, ventricular extrasystoles, as well as very rare pathogenic diseases such as arrhythmogenic right ventricular tachycardia, which can otherwise only be detected by gene sequencing.

Only an accurate resolution of the heart signal allows for this disease detection. Namely, in the case of very noisy or poorly resolved signals, the displacements of the various PQRST complexes of the heart compared to each other or over time, fluctuations in their amplitude, deformations or minor disorders are not detectable. However, this resolution of these criteria are important factors because the abovementioned problems can lead to mixing up complexes (e.g. interpreting an elevated and delayed T-wave as an R-wave, which is however common in a “healthy” heart), and to false alarms.

When a gradiometer circuit of at least two NV magnetometer units of an assembly is used, one magnetometer unit is always a greater distance from the heart (as a relatively weak magnetic field source) than another magnetometer unit. Due to the gradiometer circuit, i.e. substantially (vectorial) subtraction of what is measured, the magnetic field gradient approximately corresponds to the field originating from the weak source, while substantially stronger background fields (which are substantially the same in both magnetometer units) are eliminated. This eliminates the need for magnetic shielding so that magnetic field measurement can be carried out in everyday environments. Accordingly, the invention is particularly suitable for non-shielded measurement of weak magnetic fields. Technical details for gradiometer solutions that can also be used in the context of the present invention are disclosed in DE 102022201690.4 and are to be incorporated herein.

In one embodiment, the device is configured to sense a magnetic field strength and field direction by means of each of the NV magnetometer units of the first and/or second and/or third and/or further assembly. Another advantage of the NV sensor technology is the direction or vector information. In contrast to other technologies, this is intrinsic in NV sensor technology. Thus, interferences do not need to be introduced through modulation techniques and/or less favorable projections do not need to be used, nor do multiple separate sensors need to be used. The vector and gradiometry information is thus available at the exact same location (diamond size, i.e. single digit mm\hat{ }3 and below) and not separated by a few cm to many cm as with other technologies. With NV magnetometer units, which can determine not only the field strength but also the direction of the magnetic field, improved suppression of a background field and thus better detection of signals with superimposition of strong interference signals are enabled. This is based on two mechanisms of action: firstly, the signal is not so susceptible to tilting of the sensors with respect to one another (in a standard gradiometer application, tilting of the 1D magnetomer leads to a different projection of the measurement or interference field), and this can be compensated for, which is particularly relevant for non-rigid designs combined with localization, e.g. in a mattress. Second, the interference field may have a different spatial direction than the field being measured. This facilitates the separation of the signal and background.

In one embodiment, the device comprises a signal processing unit to which the NV magnetometer units of the first and/or second and/or third and/or further assembly are connected, wherein the device is configured by means of the signal processing unit to have an effective magnetic field strength and/or an effective magnetic field direction as a difference between the magnetic field strength and/or the magnetic field direction detected by means of the at least two NV magnetometer units. Both a wireless and wired connection are provided between the sensors and the signal processing unit.

A sampling rate which is higher than the cardiac signal, in particular greater than 50 Hz, is required for the application in order to resolve the signal. A range of 200 Hz to 400 Hz is considered to be particularly advantageous. Higher is always better for resolution, however this increases the requirements for sensitivity.

Further advantages and embodiments of the invention will emerge from the description and the accompanying drawings.

The invention is shown schematically in the drawings on the basis of exemplary embodiments and is described hereinafter with reference to the drawings.

### BRIEF DESCRIPTION OF THE DRAWINGS

FIG. 1 shows in a schematic block view the essential components of an NV-center magnetometer as may be used in the context of the invention.

FIG. 2 shows possible assemblies of NV magnetometer units of a device for detecting magnetic signals according to one embodiment in various figures a) to c), respectively in a schematic block view in each case.

FIG. 3 shows a diagram of a user with a device according to one embodiment of the invention in a side view.

FIG. 4 shows a diagram of a perspective view of a number of assemblies in a residential space embedded in different bodies according to embodiments of the invention.

### EMBODIMENT(S) OF THE INVENTION

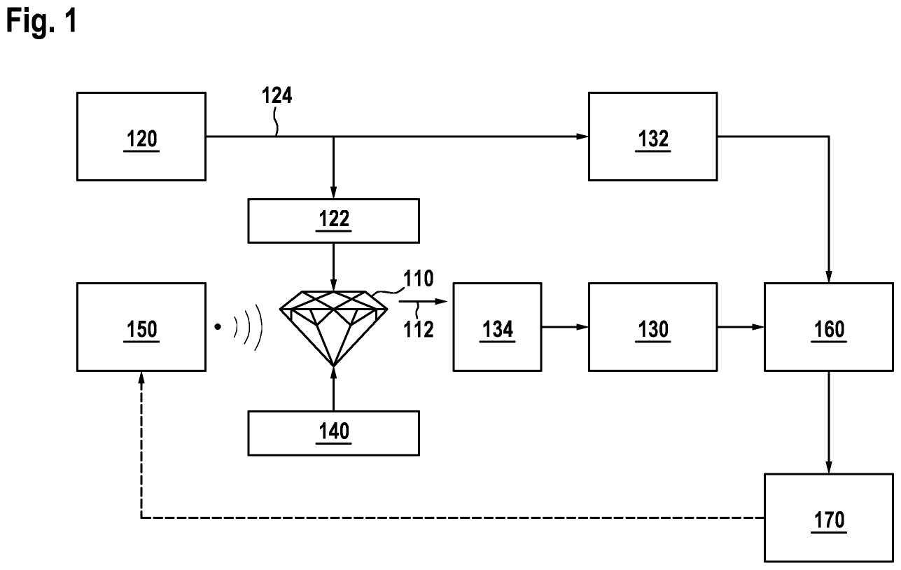

FIG. 1 shows a diagram of the essential components of an NV-center magnetometer. First, a diamond 110 with nitrogen vacancies (NV) is present as the sensor medium. The optical excitation of the NV centers is achieved by a suitable light source 120, such as an LED or a pump laser. Here, for example, a frequency-doubled Nd: YAG laser or semiconductor laser in the green range of about 510-532 nm, e.g. at 532 nm, is suitable for off-resonance excitation. Alternatively, LEDs in suitable wavelength ranges may also be used. Depending on the assembly, the light of the light source 120 may be radiated via suitable optical elements 122 such as mirrors, beam splitters, focusing optics such as lenses, and optionally via fiber optic elements in the diamonds 110. In addition, the excitation light may be irradiated continuously or in a pulsed manner by the laser such that, for example, time windows for interference-free fluorescence light measurement are kept clear.

Furthermore, a microwave source 150 may be present in the magnetometer capable of generating an electromagnetic field across a bandwidth that sufficiently covers the desired resonance frequency in the sensor medium, e.g., in the range of the NV centers in the diamonds 110. A microwave resonator structure may be used to homogeneously distribute the generated microwaves across the volume of the sensing range in the diamond. The resonator structure or microwave source 150 is preferably tuned for the frequency of the electron spin resonances. To enable vector magnetometry, an additional static bias magnetic field 140 is generated. As a result, the measurement becomes intrinsically vectorial. Different spatial directions are used in the crystal structure for this purpose. For example, to generate such a magnetic field 140, a Helmholtz coil is suitable, in which a substantially homogeneous magnetic field can be generated in a limited range by means of a pair of coils.

The resulting fluorescent light 112 from the diamond 110 may in turn be directed via suitable optical elements 134 such as optical filters, beam splitters, lenses, and/or fiber optic elements to a first photodetector 130 that is sensitive at least in the range of the fluorescence wavelength. The first photodetector 130 may also be disposed directly on the diamond 110. A second photodetector 132 is arranged to detect at least a portion of the excitation light of the light source 120, which may be decoupled by, for example, a beam splitter, a filter, or a partially permeable element. This excitation light detector signal 132 may be used as a reference signal to eliminate background signals, for example, by modulating the excitation light by way of a lock-in amplifier, and highlighting the resonance signal of interest. Additionally, or alternatively, this reference signal may be used to account for fluctuations in excitation light. Corresponding circuitry 160, such as a pre-amplifier, log amplifier, lock-in amplifier, signal filters, or others, is thus provided to receive the signals from the first and second photodetector and to pre-process the signals in an appropriate manner for further evaluation. Finally, the pre-processed fluorescence signal may be evaluated by a signal processing unit 170, e.g., with a suitable microcontroller or processor, to obtain the desired parameters of the detected magnetic field from the signal, in particular the magnetic field strength and the direction of the magnetic field.

Of course, such a device may also comprise further units not shown, such as communication units or interfaces to output measurement results. Such a device may also be advantageously integrated into an ASIC or FPGA.

In order to be usable in an everyday environment, magnetic fields not originating from desired weak sources are to be eliminated as far as possible from the measurement, in particular the Earth's magnetic field in the range of 10⁻⁵ Tesla (several microtesla). In contrast, cardiac magnetic fields range from 10 to 100 times 10⁻¹² Tesla (picotesla).

The elimination of the background magnetic fields may be achieved by a shield or a gradiometer assembly for the magnetic field measurement, according to exemplary embodiments. Gradiometers are generally referred to as sensor units that are capable of detecting not only the field strength, but also the gradient of the field.

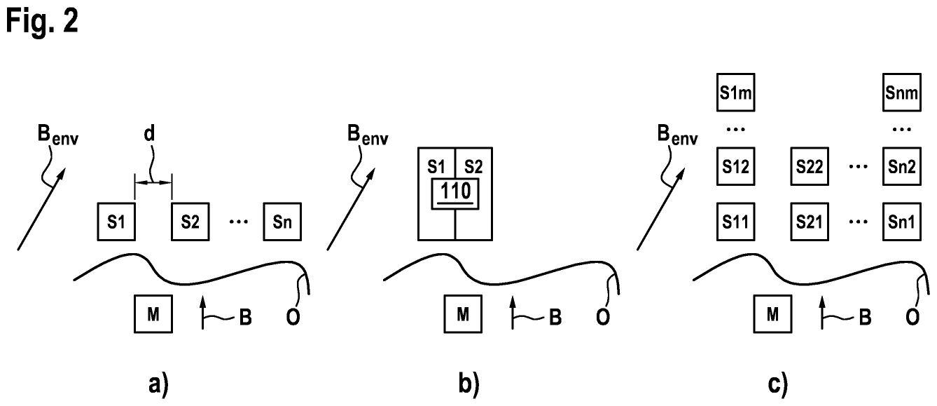

At least two individual magnetometers may be used for this purpose which are arranged at different spatial locations. As an example, a sensor unit that uses two or more NV center magnetometers in a gradiometer assembly is described below in conjunction with FIG. 2.

FIG. 2 shows possible geometric assemblies of NV magnetometer units of a device for sensing magnetic signals according to one embodiment in various figures a) to c). Figure a) shows a side view of an assembly of at least two NV magnetometer units S1, S2, . . . , Sn in any arrangement with respect to one another in a plane (perpendicular to the drawing plane, i.e. only the first row is visible). The main extension plane is thus perpendicular to the drawing plane. Figure b) shows a side view of two NV magnetometer units S1, S2 whose sensor media are a section of the same diamond crystal 110. Figure c) shows a side view of a number (n by m) of NV magnetometer units S11, S21, . . . , Sn1, S12, S22, . . . , Sn2, S1m, . . . Snm in an arrangement parallel to the drawing plane. Thus, the main extension plane is in the drawing plane.

Furthermore, M designates a signal source, here a heart, and O designates an optional surface (especially the skin of the body), which limits the accessibility to and of the magnetic field source M.

In embodiments of the inventions, more than two (but at least two) NV magnetometer units in total may form one gradiometer. With each additional NV magnetometer unit, the background field can be determined better and the location and strength of the exciter can be better separated from the background.

In other embodiments of the invention, two NV magnetometer units may also always form a gradiometer, wherein multiple gradiometers are then formed overall-depending on the number of NV magnetometer units- and the signal of interest is detected. An effective measurement signal can then be formed therefrom, in particular from the signal processing unit, for example by averaging, summation, etc.

A distance d between two NV magnetometer units S1, S2 . . . or more precisely their sensor media, corresponds to the distance between the locations where magnetic field measurements are taken simultaneously. As long as the distance between the measurement locations is relatively small, it can be assumed that the strength of an additional background magnetic field Beny is approximately equal at both locations. In contrast, the weak magnetic field B of interest will decrease significantly with increasing distance from the magnetic field source M. Furthermore, the different orientation of the magnetic field of the exciter (e.g., the heart) allows for a clear separation from the background field even at close distances.

Thus, by disposing two NV magnetometer units at different distances and angles from the source or from the heart, the background field can be eliminated or determined by vector arithmetic, thereby determining the small magnetic field of interest and characterizing its source (location and orientation). This may be further improved by a remote magnetometer unit and/or a further assembly that is so far away that the weak magnetic field of interest has fallen below the detection threshold. With such a configuration, local changes in the background field can be compensated for by the at least two near magnetometer units. For this purpose, for example, two NV magnetometer units may be arranged one above the other in an axial gradiometer configuration, such that one NV magnetometer unit of a first layer forms a gradiometer with an NV magnetometer unit located below it in a second layer below.

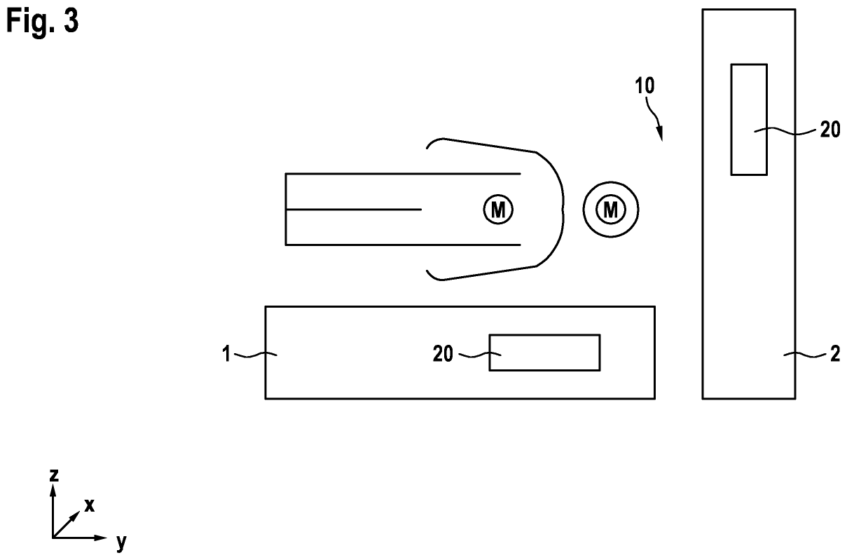

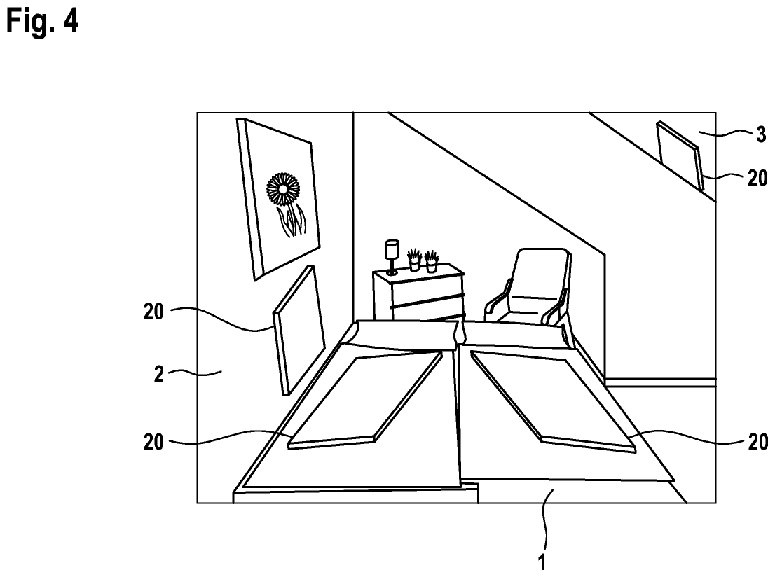

Diagrams of possible embodiments of the invention are shown in FIGS. 3 and 4, and will be described in more detail below. Like elements are provided with like reference numerals and are not described multiple times.

A device for sensing magnetic signals is shown in each case, comprising at least two assemblies 20 of at least two NV magnetometer units, each having a main extension plane. The device is used to capture magnetic signals generated by a beating heart (M), but can generally capture all magnetic signals, in particular bio-signals, i.e. those emanating from living beings, e.g. signals from a brain (M). For the purpose of illustration, the figures each have a coordinate system at the top left, wherein the drawing plane shows the x-z plane and the y-axis extends into the drawing plane. The device further comprises a signal processing unit (not shown) to which the NV magnetometer units of the assemblies 20 are connected to determine an effective magnetic field strength and/or field direction. Further, a communication unit (not shown) may be provided to connect the device to other devices such as a PC, tablet PC, and a smartphone for input, output, and operation. The communication unit may comprise, for example, wired and/or wireless interfaces. The signal processing unit and the communication unit may also be integrated in the body or arranged externally from the body, e.g. as a free-standing device, in a control panel, etc.

In FIG. 3, a mattress 1 and a wall 2 are shown as a body, in FIG. 4, a mattress 1 in a bed, a wall 2 and a roof slope 3, with an assembly 20 embedded in each of them. In addition to the above examples, embedding in any furnishings, e.g. furniture, pictures, lamps, wall coverings, etc. is contemplated.

A mattress 1, a mattress topper, a bed or a slatted bed is suitable for embedding an assembly for sensing in the frontal plane of a user.

A headboard of a bed, a wall 2, a room ceiling or a roof slope 3 is suitable for sensing the perpendicular component.

Numerous sources of magnetic ambient noise (background field) are located outside of the private living area and are thus inaccessible to a reduction. Exterior walls as well as interior walls are suitable for detection and offsetting, in particular if broadband magnetic noise fields flow through them. Due to synchronized recording, the signals of various assemblies can be offset and heart-like signals can be reduced from ambient noises.

1. A device for sensing magnetic signals generated by a beating heart, the device comprising:
a first assembly of at least two nitrogen vacancy centers, NV, magnetometer units the first assembly including a first main extension plane; and
a second assembly of at least two NV magnetometer units the second assembly including a second main extension plane,
wherein the first main extension plane is not parallel to the second main extension plane.

2. The device according to claim 1, wherein the first main extension plane and the second main extension plane include an angle of at least 25°.

3. The device according to claim 1, wherein the first main extension plane and the second main extension plane include an angle of 80°-90°.

4. The device according to claim 1, further comprising:
a third assembly of at least two NV magnetometer units, the third assembly including a third main extension plane,
wherein the third main extension plane is not parallel to the first main extension plane, and the third main extension plane is not parallel to the second main extension plane.

5. The device according to claim 4, wherein the third main extension plane and the first main extension plane and/or the second main extension plane include an angle of at least 25°.

6. The device according to claim 4, further comprising:
a further assembly of at least one NV magnetometer unit, the further assembly configured to detect a background magnetic field.

7. The device according to claim 6, wherein the first assembly, the second assembly, the third assembly, and/or the further assembly is embedded in a body selected from a building wall, a building ceiling, a building floor, and a furnishing.

8. The device according to claim 6, wherein the device is configured to detect a magnetic field strength and a field direction using each of the NV magnetometer units of the first assembly, the second assembly, the third assembly, and/or the further assembly.

9. The device according to claim 8, further comprising:
a signal processing unit with which the NV magnetometer units of the first assembly, the second assembly, the third assembly, and/or the further assembly are connected,
wherein the device is configured to measure an effective magnetic field strength and/or a field direction using the signal processing unit as a difference between magnetic field strengths and/or field directions detected using the NV magnetometer units.

10. The device according to claim 6, wherein the assembly is a two-dimensional assembly, in which the NV magnetometer units of the first assembly, the second assembly, the third assembly, and/or the further assembly are arranged in a respective main extension plane.

11. The device according to claim 4, wherein:
the NV magnetometer units of the first assembly, the second assembly, and/or the third assembly comprise at least four NV magnetometer units, and
the assembly is a three-dimensional assembly, in which at least one of the at least four NV magnetometer units is not arranged in a respective main extension plane, in which at least three other of the at least four NV magnetometer units are arranged.

12. The device according to claim 6, wherein:
each of the NV magnetometer units of the first assembly, the second assembly, the third assembly, and/or the further assembly as a sensor medium comprises a diamond crystal or a portion of a diamond crystal with nitrogen vacancy centers, and
the device is configured to sense a magnetic field strength and/or a field direction by reading a spin resonance dependent on the magnetic field strength in the sensor medium.

13. The device according to claim 12, further comprising:
at least one excitation light source configured to radiate light into the sensor medium;
at least one microwave source configured to generate a resonant field in the sensor medium; and
at least one photodetector configured to sense resonance-dependent fluorescent light from the sensor medium.

14. The device according to claim 13, wherein the NV magnetometer units of one of the first assembly, the second assembly, the third assembly, and the further assembly are associated with a same excitation light source and/or a same microwave source.

15. The device according to claim 12, wherein the sensor medium of each of at least two NV magnetometer units of one of the first assembly, the second assembly, the third assembly, and the further assembly comprises a portion of a same diamond crystal.

16. The device according to claim 12, wherein a distance between the sensor media of the at least two NV magnetometer units of one of the first assembly, the second assembly, the third assembly, and the further assembly is between 1 and 30 millimeters.
